(19) United States
(12) Patent Application Publication
(10) Pub. No.: US 2014/0308711 A1
(43) Pub. Date: Oct. 16, 2014

(54) ENZYMATIC PHOSPHOROTHIOATION OF DNA OR RNA

(71) Applicants: Massachusetts Institute of Technology, Cambridge, MA (US); Wuhan University, Wuhan (CN); Shanghai Jiaotong University, Shanghai (CN)

(72) Inventors: Peter C. Dedon, Newton, MA (US); Delin You, Shanghai (CN); Bo Cao, Shanghai (CN); Ramesh Babu Indrakanti, Belmont, MA (US); Michael S. DeMott, Newton, MA (US); Lianrong Wang, Wuhan (CN); Zixin Deng, Shanghai (CN); Xiufen Zhou, Shanghai (CN)

(73) Assignees: Massachusetts Institute of Technology, Cambridge, MA (US); Wuhan University, Wuhan (CN); Shanghai Jiaotong University, Shanghai (CN)

(21) Appl. No.: 14/210,188

(22) Filed: Mar. 13, 2014

Related U.S. Application Data
(60) Provisional application No. 61/782,236, filed on Mar. 14, 2013.

(51) Int. Cl. C12N 15/64; C12N 9/10

(52) U.S. Cl. CPC C12N 15/64; C12N 9/13
USPC 435/91.4; 435/193; 435/283.1

(57) ABSTRACT

This invention relates to methods, compositions, and kits for enzymatic phosphorothioation of the sugar-phosphate backbone of nucleic acids. The invention allows for phosphorothioation of pre-existing nucleic acids.

### RELATED APPLICATIONS

This application claims the benefit of the filing date of U.S. Provisional Application No. 61/782,236, filed Mar. 14, 2013, the entire contents of which are incorporated by reference herein.

### GOVERNMENT SUPPORT

This invention was made with government support under Grant No. CHE1019990 awarded by the National Science Foundation. The government has certain rights in the invention.

### FIELD OF INVENTION

This invention relates to compositions and methods for enzymatic phosphorothioation of the sugar-phosphate backbone of nucleic acids.

### BACKGROUND OF INVENTION

Phosphorothioation of the DNA and RNA backbone has been shown to confer resistance to degradation of the nucleic acids by nucleases and has thus been recognized as a means to stabilize oligonucleotides for delivery in gene therapy, siRNA, and other applications. Existing methods to incorporate phosphorothioate linkages utilize chemically modified precursors that are inserted either during solid phase nucleic acid synthesis in the case of oligonucleotides, or during microbial production in the case of larger nucleic acid molecules, including plasmids. However, these methods preclude the incorporation of phosphorothioate linkages into large synthetic DNA and RNA molecules that are difficult to synthesize, or when the DNA or RNA molecules have already been synthesized or purified.

Accordingly, there exists a need for methods and compositions that allow for the incorporation of phosphorothioate modifications into nucleic acids of any size, including pre-existing nucleic acids.

### SUMMARY OF INVENTION

The present invention provides methods, compositions, and kits for incorporating phosphorothioate modifications into nucleic acids. In particular, the present invention addresses problems that exist with current technologies that utilize artificial or microbial means of synthesizing phosphorothioated nucleic acids de novo. Such problems, as described herein, to include an inability to synthesize large phosphorothioate modified nucleic acids, the inability to synthesize phosphorothioate modified nucleic acids having purely the Rp stereochemical configuration, and the inability to incorporate phosphorothioate modifications into pre-existing nucleic acid molecules.

Thus, the present invention relates to the surprising discovery that certain bacteria contain gene clusters capable of incorporating phosphorothioate modifications into nucleic acids, such as existing nucleic acids. As described herein, these genes or gene clusters are typically denoted the dnd genes, or the dnd gene cluster. These genes or gene clusters refer to those that code for proteins that can phosphorothioate nucleic acids as provided herein. These proteins include homologs or equivalents that retain the ability to phosphorothioate alone or in combination with other Dnd proteins or homologs or equivalents thereof. Accordingly, any recitation of dnd gene or Dnd proteins can alternatively refer to the homolog or equivalent in any one of the methods, compositions or kits provided herein. It is well within the skill of those of ordinary skill in the art to be able to identify the homologs and equivalents that can be included in the compositions or kits provided herein or used in the methods provided herein.

Accordingly, in one aspect, methods for incorporating phosphorothioate modifications into the sugar-phosphate backbone of a nucleic acid are provided. In one embodiment of any one of the methods provided, the method comprises transforming a bacterium, which expresses a one or more Dnd proteins that incorporate phosphorothioate modifications into a nucleic acid, with a nucleic acid vector to be modified, and isolating the vector from the bacterium. In another embodiment of any one of the methods provided, the bacterium is Escherichia coli. In another embodiment of any one of the methods provided, the bacterium expresses the Dnd proteins endogenously or is engineered to express the Dnd proteins. Accordingly, in another embodiment of any one of the methods provided, the bacterium expresses the Dnd proteins from either a plasmid or from the bacterium's genomic DNA. In another embodiment of any one of the methods provided, the bacterium expresses one or more Dnd proteins sufficient to phosphorothioate a nucleic acid. In another embodiment of any one of the methods provided, the bacterium expresses DndA, DndC, DndD, DndE, and optionally DndB, or homologs or equivalents thereof. In another embodiment of any one of the methods provided, the Dnd proteins are those of dnd genes from a genus selected from the group consisting of Bacillus, Burkholderia, Candidatus Methanoregula, Candidatus Pelagibacter, Citrobacter, Clostridium, Desulfatibacillum, Enterobacter, Escherichia, Exiguobacterium, Geobacter, Hahella, Mesorhizobium, Mycobacterium, Oceanobacter, Pseudoalteromonas, Pseudomonas, Roseobacter, Salmonella, Shewanella, Streptomyces, Vibrio, and combinations thereof. In another embodiment of any one of the methods provided, the Dnd proteins are from dnd genes from a species selected from the group consisting of Streptomyces lividans, Salmonella enterica, Pseudomonas fluorescens, Escherichia coli, and combinations thereof. In another embodiment of any one of the methods provided, the bacterium is contacted with Dnd enzyme co-factors and substrates. In another embodiment of any one of the methods provided, the vector is a plasmid between 1 and 25 kb in length. In another embodiment of any one of the methods provided, the phosphorothioate modifications are of the Rp stereo-isoform configuration. In another embodiment of any one of the methods provided, the vector is isolated after an amount of time sufficient for the incorporation of phosphorothioate modifications.

According to another aspect, a composition for incorporating phosphorothioate modifications into the sugar-phosphate backbone of a nucleic acid is provided. In one embodiment of any one of the compositions provided, the composition comprises a bacterial lysate, the lysate comprising one or more Dnd proteins that incorporate phosphorothioate modifications into a nucleic acid. In another embodiment of any one of the compositions provided, the bacterial lysate comprises DndA, DndC, DndD, DndE, and optionally DndB, or homologs or equivalents thereof. In another embodiment of any one of the compositions provided, the bacterial lysate comprises Dnd proteins expressed from dnd genes from a genus selected from the group consisting of Bacillus, Burkholderia, Candidatus Methanoregula, Candidatus Pelagibacter, Citrobacter, Clostridium, Desulfatibacillum, Enterobacter, Escherichia, Exiguobacterium, Geobacter, Hahella, Mesorhizobium, Mycobacterium, Oceanobacter, Pseudoalteromonas, Pseudomonas, Roseobacter, Salmonella, Shewanella, Streptomyces, Vibrio and combinations thereof. In another embodiment of any one of the compositions provided, the bacterial lysate comprises Dnd proteins expressed from dnd genes from a species selected from the group consisting of Streptomyces lividans, Salmonella enterica, Pseudomonas fluorescens, Escherichia coli, and combinations thereof. In another embodiment of any one of the compositions provided, the composition further comprises Dnd enzyme co-factors and substrates.

In another embodiment of any one of the compositions provided, the composition comprises one or more purified Dnd proteins that incorporate phosphorothioate modifications into a nucleic acid. In one embodiment of any one of the compositions provided, the purified

Dnd proteins comprise DndA, DndC, DndD, DndE, and optionally DndB, or homologs or equivalents thereof. In another embodiment of any one of the compositions provided, the purified Dnd proteins are expressed from dnd genes from a genus selected from the group consisting of Bacillus, Burkholderia, Candidatus Methanoregula, Candidatus Pelagibacter, Citrobacter, Clostridium, Desulfatibacillum, Enterobacter, Escherichia, Exiguobacterium, Geobacter, Hahella, Mesorhizobium, Mycobacterium, Oceanobacter, Pseudoalteromonas, Pseudomonas, Roseobacter, Salmonella, Shewanella, Streptomyces, Vibrio and combinations thereof. In another embodiment of any one of the compositions provided, the purified Dnd proteins are expressed from dnd genes from a species selected from the group consisting of Streptomyces lividans, Salmonella enterica, Pseudomonas fluorescens, Escherichia coli, and combinations thereof. In another embodiment of any one of the compositions provided, the composition further comprises Dnd enzyme co-factors and substrates.

In another aspect, a method for incorporating phosphorothioate modifications into the sugar-phosphate backbone of a nucleic acid is provided. In one embodiment of any one of the methods provided, the method comprises contacting a nucleic acid with any of the compositions provided herein, and isolating the nucleic acid. In another embodiment of any one of the methods provided, the nucleic acid is isolated after an amount of time sufficient for the incorporation of phosphorothioate modifications.

In another aspect, a kit for incorporating phosphorothioate modifications into the sugar-phosphate backbone of a nucleic acid is provided. In one embodiment of any one of the kits provided, the kit comprises any one or more of the compositions provided herein. In another embodiment of any one of the kits provided, the kit comprises a transformable bacterium which expresses one or more Dnd proteins that incorporate phosphorothioate modifications into a nucleic acid, and optionally directions for use. In another embodiment of any one of the kits provided, the bacterium is Escherichia coli. In another embodiment of any one of the kits provided, the bacterium expresses the one or more Dnd proteins endogenously or as a result of being engineered to do so. Accordingly, in one embodiment of any one of the kits provided, the one or more Dnd proteins are expressed from either a plasmid or from the bacterium's genomic DNA. In another embodiment of any one of the kits provided, the bacterium expresses DndA, DndC, DndD, DndE, and optionally DndB, or homologs or equivalents thereof. In another embodiment of any one of the kits provided, the Dnd proteins are expressed from dnd genes to from a genus selected from the group consisting of Bacillus, Burkholderia, Candidatus Methanoregula, Candidatus Pelagibacter, Citrobacter, Clostridium, Desulfatibacillum, Enterobacter, Escherichia, Exiguobacterium, Geobacter, Hahella, Mesorhizobium, Mycobacterium, Oceanobacter, Pseudoalteromonas, Pseudomonas, Roseobacter, Salmonella, Shewanella, Streptomyces, Vibrio, and combinations thereof. In another embodiment of any one of the kits provided, the Dnd proteins are expressed from dnd genes from a species selected from the group consisting of Streptomyces lividans, Salmonella enterica, Pseudomonas fluorescens, Escherichia coli, and combinations thereof. In another embodiment of any one of the kits provided, the kit further comprises Dnd enzyme co-factors and substrates. In another embodiment of any one of the kits provided, the kit further comprises one or more containers, wherein at least one container comprises the bacterium.

In another aspect, a kit for incorporating phosphorothioate modifications into the sugar-phosphate backbone of a nucleic acid comprises one or more purified Dnd proteins (or a bacterial lysate comprising one or more Dnd proteins) that incorporate phosphorothioate modifications into a nucleic acid, and optionally directions for use. In one embodiment of any one of the kits provided, the Dnd proteins comprise DndA, DndC, DndD, DndE, and optionally DndB, or homologs or equivalents thereof. In another embodiment of any one of the kits provided, the Dnd proteins are expressed from dnd genes from a genus selected from the group consisting of Bacillus, Burkholderia, Candidatus Methanoregula, Candidatus Pelagibacter, Citrobacter, Clostridium, Desulfatibacillum, Enterobacter, Escherichia, Exiguobacterium, Geobacter, Hahella, Mesorhizobium, Mycobacterium, Oceanobacter, Pseudoalteromonas, Pseudomonas, Roseobacter, Salmonella, Shewanella, Streptomyces, Vibrio and combinations thereof. In another embodiment of any one of the kits provided, the Dnd proteins are expressed from dnd genes from a species selected from the group consisting of Streptomyces lividans, Salmonella enterica, Pseudomonas fluorescens, Escherichia coli, and combinations thereof. In another embodiment of any one of the kits provided, the kit further comprises Dnd enzyme co-factors and substrates. In another embodiment of any one of the kits provided, the kit further comprises one or more containers, wherein at least one container comprises one or more Dnd proteins.

In another aspect, a kit for incorporating phosphorothioate modifications into the sugar-phosphate backbone of a nucleic acid comprises one or more vectors comprising one or more dnd genes. In one embodiment of any one of the kits provided, the one or more vectors to comprise dndA, dndC, dndD, dndE, and optionally dndB, or homologs or equivalents thereof. In another embodiment of any one of the kits provided, the one or more vectors comprise dnd genes from a genus selected from the group consisting of Bacillus, Burkholderia, Candidatus Methanoregula, Candidatus Pelagibacter, Citrobacter, Clostridium, Desulfatibacillum, Enterobacter, Escherichia, Exiguobacterium, Geobacter, Hahella, Mesorhizobium, Mycobacterium, Oceanobacter, Pseudoalteromonas, Pseudomonas, Roseobacter, Salmonella, Shewanella, Streptomyces, Vibrio, and combinations thereof. In another embodiment of any one of the kits provided, the one or more vectors comprise dnd genes from a species selected from the group consisting of Streptomyces lividans, Salmonella enterica, Pseudomonas fluorescens, Escherichia coli, and combinations thereof. In another embodiment of any one of the kits provided, the kit further comprises bacteria that can be transformed with the one or more vectors. In another embodiment of any one of the kits provided, the kit further comprises Dnd enzyme co-factors and substrates. In another embodiment of any one of the kits provided, the kit further comprises one or more containers, wherein at least one container comprises the one or more vectors.

In another embodiment of any one of the methods, compositions or kits provided, a step (or the means for doing so) of adding consensus sequences recognized by the product(s) of the dnd gene clusters, or homologs or equivalents thereof, to the nucleic acids to be modified can be further included.

In another embodiment of any one of the methods, compositions or kits provided, the Dnd protein(s) is/are a protein/proteins that can phosphorothioate nucleic acids alone or in combination with other Dnd protein(s) (or homolog(s) or equivalent(s) thereof).

In another embodiment of any one of the methods, compositions or kits provided, a dnd gene or Dnd protein may be a homolog or equivalent as provided herein or otherwise known or identifiable by one of ordinary skill in the art. For instance, in one embodiment of any one of the methods, compositions or kits provided DndA may be instead the equivalent IscS protein or the dnd gene may be the gene that encodes IscS protein.

The subject matter of this application may involve, in some cases, interrelated products, alternative solutions to a particular problem, and/or a plurality of different uses of a single system or article.

Other advantages, features, and uses of the invention will be apparent from the detailed description of certain non-limiting embodiments, the drawings, which are schematic and not intended to be drawn to scale, and the claims.

### BRIEF DESCRIPTION OF THE DRAWINGS

FIG. 1 depicts graphs of LC-MS/MS data demonstrating the detection of phosphorothioate (PT)-linked dinucleotides synthesized in vitro in reactions of duplex oligodeoxynucleotide substrates with cell-free extracts from S. enterica. (A, B) Calibration of the LC-MS/MS system using synthetic PT-containing dinucleotide standards (A) d(G_{PS}A) and (B) d(G_{PS}T) standards. (C) Positive control using oligodeoxynucleotides dpt101 to demonstrate nuclease hydrolysis and LC-MS/MS detection of PT-containing dinucleotides. (D) Reactions of cell-free extracts with duplex oligodeoxynucleotide substrate dpt 102. (E) Negative control lacking an oligodeoxynucleotide substrate in the reaction system. (F) Negative control using a cell-free extract prepared from XTG103 mutant cells lacking dnd genes.

FIG. 2 depicts graphs of LC-MS/MS data demonstrating the determination of the consensus sequence context for PT modifications generated using cell-free extracts from S. enterica. (A) Duplex dpt 104 as substrate. (B) PT-containing dinucleotides d(G_{PS}A) and d(G_{PS}T) as standards. (C) Duplex dpt 105 as substrate.

FIG. 3 depicts graphs of LC-MS/MS data demonstrating that flanking sequence does not affect the incorporation of PT modifications at the GAAC/GTTC consensus in reactions of cell-free extracts with oligodeoxynucleotides substrates. (A) Duplex dpt 106 as substrate. (B) Duplex dpt 107 as substrate. (C) Reaction of a substrate containing a single PT modification of the GAAC strand of a duplex substrate (duplex dpt113). (D) Reaction of a substrate containing a single PT modification of the GTTCC strand of a duplex substrate (duplex dpt114). (E) The complimentary strand of dpt113 as a single-stranded substrate; (F) The complimentary strand of dpt114 as a single-stranded substrate.

FIG. 4 depicts SDS-PAGE data of purified Dnd proteins, and graphs of LC-MS/MS data demonstrating that PT modifications are incorporated in vitro by purified recombinant Dnd proteins. Top left panels: SDS-PAGE gels showing purified His-tagged IscS protein (A1, right lane) and His-tagged DndCDE complex (A2, left two lanes with different amounts of protein loaded in each lane); size markers are shown in the left and right lanes of panels A1 and A2, respectively. LC-MS analysis of PT-containing dinucleotides in pBluescript SK+ plasmid (B) and 60 bp double-stranded oligodeoxynucleotides containing GAAC/GTTC consensus sequences with different flanking sequences: dpt116 (C) and dpt117 (D).

### DETAILED DESCRIPTION OF INVENTION

Phosphorothioate modifications positioned in DNA or RNA confer resistance to nuclease degradation and can, therefore, be useful in therapeutic, diagnostic or other applications in which degradation is an impediment. For example, antisense oligonucleotides have therapeutic applications due to their ability to interfere with gene expression in a sequence-specific manner. However, typical oligonucleotides are degraded or metabolized quickly, diminishing their effectiveness in the context of treatment. Thus, as phosphorothioate modified oligonucleotides are considerably more stable against nuclease degradation compared to their phosphodiester counterparts, they are generally considered to be better therapeutic candidates. Indeed, the first FDA approved antisense drug in the United States, VITRAVENE, is a phosphorothioate modified oligonucleotide.

Currently, incorporation of phosphorothioate modifications involves chemical synthesis of modified nucleic acids, or limited microbial means that catalyze the in vivo synthesis of modified plasmids (e.g., via polymerase based enzymatic polymerization of phosphorothioate nucleotide analogs). These methods preclude the incorporation of phosphorothioate linkages into large synthetic nucleic acid molecules that are too large to synthesize, and are not amenable for incorporating phosphorothioate modifications into nucleic acid molecules that have already been synthesized or purified. Additionally, these methods generally do not control for the stereospecificity of the modification, as typical racemic mixtures arise during solid phase chemical synthesis of phosphorothioated nucleic acids Importantly, there is evidence that siRNA oligonucleotides possessing phosphorothioates in which the sulfur atom exclusively adopts an Rp orientation are better able to interact with the target messenger RNA and thus lead to more rapid and specific degradation of the duplex by RNase H.

Accordingly, the present invention addresses the above problems through the surprising discovery that phosphorothioate modifications can be added, even stereospecifically in some embodiments, by enzymes of any of the various bacterial dnd genes clusters (or homologs or equivalents thereof), into nucleic acids, such as pre-existing nucleic acids. The system can provide direct enzymatic modification to e.g., a DNA or RNA molecule, with application to molecules of any size or origin. The dnd gene products can insert only the Rp configuration that has been shown to confer beneficial properties in many applications, in some embodiments. Thus, the present invention allows broad application of the stability and protection afforded by phosphorothioation of nucleic acids, and it overcomes the insufficiencies of purely chemical methods of synthesis.

### The dnd Genes/Gene Products

The dnd gene cluster, which generally codes for five proteins (e.g., DndA, DndB, DndC, DndD, and DndE), was originally discovered in the genus Streptomyces where it was found to be responsible for a DNA degradation phenotype (the Dnd phenotype) apparent during agarose gel electrophoresis with Tris buffer (Zhou et al., Mol. Microbiol. 2005, 57(5):1428-38). The presence of phosphorothioate linkages, something never before seen in living cells, represents a post-synthetic modification inserted by products of the dnd gene cluster (or homologs or equivalents thereof). Distribution of the sulfur modifications was found to be sequence selective, depending on the specific bacterial strain analyzed. Analyses of different bacteria revealed phosphorothioate (PS) modifications in virtually every possible sequence context, including, but not limited to, d(G_{PS}A), d(G_{PS}T), d(G_{PS}G), d(C_{PS}A), d(A_{PS}A), d(T_{PS}A), d(C_{PS}C), d(A_{PS}C) and d(T_{PS}C).

The dnd gene cluster has also been cloned from a species in the genus Salmonella. When introduced and recombinantly expressed in bacteria, e.g., Escherichia coli, the Salmonella dnd genes produced phosphorothioate linkages in the E. coli genome (Wang et al., Nat Chem Biol. 2007, 3(11):709-10). Generally, the proteins generated by the dnd gene cluster (or homologs or equivalents thereof) are sufficient, although, in some embodiments, small molecule biochemical co-factors can be introduced (e.g., L-cysteine as a sulfur donor, pyridoxal phosphate as a co-factor, divalent metal ions, etc.), to generate phosphorothioate modifications in nucleic acids, such as the DNA of an organism devoid of a naturally occurring pathway for this modification.

As an example, Salmonella enterica serovar Cerro 87 possesses a PT modification system comprised of IscS protein (in place of DndA) and Dnd proteins B, C, D, and E. DndB is a transcriptional regulator that is generally not required for the PT reaction in some embodiments. In the context of S. enterica, an IscS protein may be considered an equivalent of DndA. Thus, the PT modification system of S. enterica may comprise IscS, DndC, DndD, and to DndE. S. enterica proteins catalyze incorporation of PT into the d(G_{PS}A) and d(G_{PS}T) contexts in a 1:1 ratio. This pattern of incorporation indicates that the consensus sequence for modification comprises GAAC on one strand and GTTC on the other as a palindromic PT consensus sequence. For example, this consensus sequence may be incorporated into any nucleic acid construct, as provided herein, that is used as a substrate for PT modification reactions. PT modification reactions may be conducted in vivo or in vitro.

Additionally, mutational analyses and functional characterization of the dnd genes has demonstrated that four of the five genes are generally needed for the PS modification. For example, mutagenesis studies demonstrated that dndA, dndC, dndD (spfD in P. fluorescens Pf0-1), and dndE are required for the DNA degradation phenotype, and thus PS incorporation (Zhou et al., Mol. Microbiol. 2005, 57(5):1428-38; Xu et al., BMC Microbiol. 2009, 9, 41; Yao et al., FEBS Lett. 2009, 583, 729-733). Conversely, dndB mutants demonstrated a significantly aggravated Dnd phenomenon (Liang et al., Nucleic Acids Res. 2007, 35, 2944-2954; Xu et al., BMC Microbiol. 2009, 9, 41), resultant from increased phosphorothioation and altered sequence specificity.

The dndA gene product is a cysteine desulfurase which is involved in the first step of DNA phosphorothioation (Chen et al., PLoS One. 2012, 7(5):e36635). DndB is predicted to be a DNA topology-modifying protein, like a DNA gyrase, and thus affects the efficiency and/or specificity of PS modification (Liang et al., Nucleic Acids Res. 2007, 35, 2944-2954). DndC contains a [4Fe-4S] cluster and shows observable ATP pyrophosphatase activity, catalyzing hydrolysis of ATP to AMP and pyrophosphate (Zhou et al., Mol. Microbiol. 2005, 57(5):1428-38; You et al., Biochemistry. 2007, 46, 6126-6133). DndD shares homology with the ATP-binding cassette (ABC) ATP-binding proteins, has ATP-ase activity, and is believed to provide the energy for stabilizing DNA secondary structures and/or nicking the DNA during the modification process by hydrolyzing ATP (Zhou et al., Mol. Microbiol. 2005, 57(5):1428-38; Yao et al., FEBS Lett. 2009, 583, 729-733; Chen et al., Protein Cell. 2010, 1(1):14-21). Lastly, DndE has 46% identity to phosphoribosylaminoimidazole carboxylase (NCAIR synthetase) from Anabaena variabilis ATCC 29413, which is known to act at a condensing carboxylation step in purine biosynthesis (Nakamura et al., DNA Res. 2002, 9, 123-130; Chen et al., Protein Cell. 2010, 1(1):14-21). DndE has also been shown to bind to nicked DNA (Hu et al., Cell Res. 2012, 22(7), 1203-1206), which may occur following DNA nicking by DndD (Zhou et al., Mol. Microbiol. 2005, 57(5):1428-38; Yao et al., FEBS Lett. 2009, 583, 729-733; Chen et al., Protein Cell. 2010, 1(1):14-21).

As described herein, dnd gene products of the present invention constitute a system capable of performing PS modifications with significant advantages over currently employed methodology. There is virtually limitless potential for site-specific PS incorporation by, for example, cloning the dnd genes from any organism harboring them, adding or removing dndB from a system, or combining different dnd homologs, or equivalents thereof, from various species to effectuate varying amounts of PS modifications at desired sequences. Accordingly, in another embodiment of any one of the methods, compositions or kits provided, the proteins for phosphorothioation can all be from the same species or they can be from different species provided they can phosphorothioate when combined.

### Compositions for Phosphorothioate (PS) Modification of Nucleic Acids

According to certain embodiments of the invention, compositions for incorporating phosphorothioate (PS) modifications into the sugar-phosphate backbone of a nucleic acid are provided. As used herein, “nucleic acid” refers to polynucleotides or oligonucleotides such as deoxyribonucleic acid (DNA, e.g., oligodeoxyribonucleotides) and ribonucleic acid (RNA, e.g., oligoribonucleotides). The term should also be understood to include, as applicable to the embodiment being described, single-stranded (such as sense or antisense) and double stranded polynucleotides, including double-stranded DNA-RNA hybrids. The term “nucleic acid” also is synonymous, and may be used interchangeably with the term “nucleic acid molecule.” Additionally, “phosphorothioate modification,” “PT modification, or “PS modification,” may be used interchangeably with “phosphorothioate linkage,” and generally refers to a modification of the sugar-phosphate backbone of a nucleic acid in which one of the non-bridging oxygens is replaced by a sulfur atom. In some aspects, a “PS” modification or linkage may also be referred to as a “PT” modification or linkage. As described herein, PS modifications can exist in either the Rp or Sp stereo-isoform configuration, as depicted below:

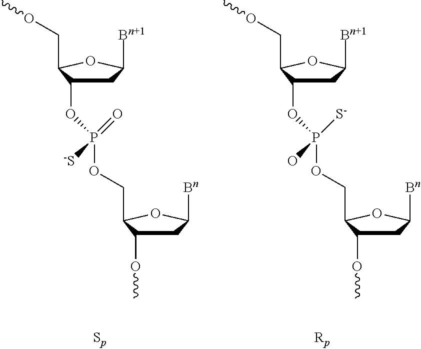

In another embodiment of any one of the methods, compositions or kits provided, one or more phosphorothioate modifications occurs or can occur as a result of the method or use of the composition or kit. In another embodiment of any one of the methods, the nucleic acid for modification can be single-stranded or double-stranded. In another embodiment of any one of the methods, the nucleic acid for modification can be a hemi-modified double-stranded nucleic to acid.

In some aspects, methods and compositions (including kits) are provided for incorporating PS modifications into a nucleic acid which are exclusively of the Rp configuration, which has advantageous effects in certain applications as described herein. Compositions comprising nucleic acids with modifications that are exclusively of the Rp configuration are also provided. For example, the compositions or methods provided herein can be amenable for incorporating PS modifications into a nucleic acid which are exclusively of the Rp configuration.

In some embodiments, bacteria that express dnd genes (or homologs or equivalents) are provided. In some aspects, the bacteria express all five dnd genes, or homologs or equivalents thereof. For example, the invention provides bacteria which express dndA, dndB, dndC, dndD, and dndE. In other aspects, provided bacteria express one or more dnd genes, or dnd proteins, sufficient to incorporate PS modifications into a nucleic acid. For example, in some embodiments, the bacteria do not express dndB for incorporating PS modifications, provided the bacteria express a number of dnd genes, or Dnd proteins, sufficient to incorporate PS modifications. In some embodiments, such bacteria express dndA, dndC, dndD, and dndE (or homologs or equivalents thereof). In some aspects, the bacteria express dnd genes isolated from Streptomyces lividans, for example as described in Zhou et al., Mol. Microbiol. 2005. In some aspects, the bacteria express dnd genes isolated from a species selected from the group consisting of Streptomyces lividans, Salmonella enterica, Pseudomonas fluorescens, Escherichia coli, and combinations thereof.

In some embodiments, provided bacteria may express one or more dnd genes isolated from one species, and also express one or more dnd genes isolated from one or more other species. In other aspects, provided bacteria express dnd genes isolated from any organism capable of incorporating PS modifications. For example, organisms known to contain dnd genes include, but are not limited to, organisms belonging a following genera: Bacillus, Burkholderia, Candidatus Methanoregula, Candidatus Pelagibacter, Citrobacter, Clostridium, Desulfatibacillum, Enterobacter, Escherichia, Exiguobacterium, Geobacter, Hahella, Mesorhizobium, Mycobacterium, Oceanobacter, Pseudoalteromonas, Pseudomonas, Roseobacter, Salmonella, Shewanella, Streptomyces, and Vibrio. In some aspects, provided bacteria express dnd genes isolated from genetic elements (defined by NCBI RefSeq accession number) of the following organisms: Bacillus cereus E33L (NC_{—}006274), Burkholderia ambifaria MC40-6 (NC_{—}010551), Burkholderia ambifaria MC40-6 (NC_{—}010551), Candidatus Pelagibacter ubique HTCC1002 (NZ_AAPV01000002), Citrobacter koseri ATCC BAA-895 (NC_{—}009792), Clostridium botulinum E3 str. Alaska E43 (NC_{—}010723), Clostridium perfringens NCTC 8239 (NZ_ABDY01000007), Desulfatibacillum alkenivorans AK-01 (NZ_ABII01000002), Enterobacter sp. 638 (NC009436), Escherichia coli B7A (NZ_AAJT01000066), Exiguobacterium sp. AT1b (NZ_ABPF01000011), Geobacter uraniireducens Rf4 (NC_{—}009483), Hahella chejuensis KCTC 2396 (NC_{—}007645), Mesorhizobium sp. BNC1 Plasmid 3 (NC_{—}008244), Mycobacterium abscessus ATCC 19977 (NC_{—}010397), Oceanobacter sp. RED65 (NZ_AAQH01000003), Pseudoalteromonas haloplanktis TAC125 chromosome II (NC_{—}007482), Pseudomonas fluorescens PfO-1 (NC_{—}007492), Roseobacter denitrificans OCh 114 (NC_{—}008209), Salmonella enterica serovar Saintpaul SARA23 (NZ_ABAM01000005), Shewanella pealeana ATCC 700345 (NC_{—}009901), Streptomyces avermitilis MA-4680 (NC_{—}003155), Streptomyces lividans 1326 (EF210454), Vibrio cholerae MZO-2 (NZ_AAWF01000002), Vibrio cholerae MZO-3 (NZ_AAUU01000003), and Vibrio fischeri MJ11 chromosome II (NC_{—}011186). In some aspects, provided bacteria express any combination of dnd genes described herein, for example, any combination of one or more dnd genes from any organism that is capable of incorporating PS modification into nucleic acids, or homologs or equivalents thereof, as described herein. In some aspects, provided bacteria express certain one or more endogenous, or wild type dnd genes, while expressing one or more dnd genes from another organism.

In another aspect, provided bacteria express any combination of dnd genes described herein, wherein one or more of the dnd genes is mutated (provided the bacteria are still able to phosphorothioate as provided herein alone or in combination with one or more other dnd proteins). By “mutated,” it is meant that a particular gene (e.g., dndA, dndB, dndC, dndD, dndE, and combinations thereof) contains one or more changes in a wild type or naturally-occurring nucleotide sequence that result in one or more amino acid substitutions, additions, or in some cases, deletions or truncations, in the protein expressed therefrom. Mutations include, but are not limited to, nucleotide substitutions, insertions, deletions (including truncations), or combinations thereof. For example, certain conservative amino acid substitutions are contemplated. Conservative amino acid substitutions are amino acid substitutions in which the substituted amino acid residue is of similar charge as the replaced residue and/or is of similar or smaller size than the replaced residue. Conservative substitutions of amino acids include substitutions made amongst amino acids within the following groups: (a) the small non-polar amino acids, A, M, I, L, and V; (b) the small polar amino acids, G, S, T and C; (c) the amido amino acids, Q and N; (d) the aromatic amino acids, F, Y and W; (e) the basic amino acids, K, R and H; and (f) the acidic amino acids, E and D. Substitutions which are charge neutral and which replace a residue with a smaller residue may also be considered conservative substitutions even if the residues are in different groups (e.g., replacement of phenylalanine with the smaller isoleucine). Methods for making amino acid substitutions, additions, or deletions are well known in the art, e.g., polymerase chain reaction (PCR)-directed methods (Molecular Biology: Current Innovations and Future Trends. by Griffin A. M. and Griffin H. G. (1995) Horizon Scientific Press, Norfolk, U.K; Modern Genetic Analysis. by Griffith A. J., Second Edition, (2002) H. Freeman and Company, New York, N.Y.). In some aspects, the mutant dnd genes or proteins are substantially homologous to their wild type counterparts. The phrase “substantially homologous” in the context of two nucleic acids or polypeptides, generally refers to two or more sequences or subsequences that have at least 40%, 60%, 80%, 90%, 95%, 98% nucleotide or amino acid residue identity, when compared and aligned for maximum correspondence, as measured using known sequence comparison algorithms (e.g., ClustalW2) or by visual inspection. The substantial homology, or identity, can exist over a region of the sequences that is at least about 50 residues in length, such as over a region of at least about 100 residues, or over a region of at least about 150 residues. In some aspects, the mutations affect e.g., the sequence specificity, the rate of reaction, the frequency of incorporation, or combinations thereof, with respect to PS modifications. In embodiments, the homologs and equivalents include those with the foregoing mutations or have the foregoing homology to a dnd gene or protein expressed therefrom, provided the homolog or equivalent can phosphorothioate in combination with other dnd gene products.

According to one embodiment, compositions for incorporating phosphorothioate modifications into the sugar-phosphate backbone of a nucleic acid are provided. In some aspects, the composition comprises a bacterial lysate. In some aspects, the “bacterial lysate” is also referred to as a “cell-free extract.” The bacterial lysate is prepared from bacteria expressing one or more Dnd proteins (or homologs or equivalents as provided herein) sufficient to incorporate phosphorothioate modifications into a nucleic acid (e.g., as described herein) alone or in combination with other Dnd proteins (or homologs or equivalents thereof), for example any bacteria provided herein. The lysate can be prepared by lysing bacterial cells using any convenient method that substantially maintains enzyme activity, e.g., sonication, French press, and the like as known in the art. The lysate may be fractionated, particulate matter spun out, etc., or may be used in the absence of additional processing steps. The cell lysate may be further combined with substrates, co-factors and such salts, metal ions, buffers, etc., as may be needed for enzyme activity. For example, the composition may further comprise e.g., L-cysteine as a sulfur donor, pyridoxal phosphate as a co-factor for Dnd enzyme activity, divalent metal ions, buffers to control pH, salts to control ionic strength, etc.

In some aspects, the composition comprises one or more purified Dnd proteins (or homologs or equivalents thereof) sufficient to incorporate phosphorothioate modifications into a nucleic acid alone or in combination with other Dnd proteins (or homologs or equivalents thereof), e.g., as described herein. In some aspects, the proteins are recombinantly expressed and purified according to known methods, for example as described in Kingston and Brent, Current Protocols in Molecular Biology: Ch. 16 Protein Expression, John Wiley & Sons, Inc. (2007). In some aspects a “purified protein” is a protein isolated from a cell or cell lysate according to known methods, for example, those described by Kingston and Brent, supra. In some aspects, the purified protein is combined with one or more other purified proteins, e.g., one or more purified Dnd proteins as described herein. In some aspects, a “purified” protein or protein faction, is “substantially pure,” meaning the protein (or group of proteins, e.g., one or more Dnd proteins) is (are) the predominant species present (e.g., on a molar basis, more abundant than any other individual macromolecular species in the composition), and a substantially purified fraction of the protein is a composition wherein the protein comprises at least about 50%, at least about 60%, at least about 70%, at least about 80%, at least about 90%, at least about 95%, or at least about 99% (on a molar basis) of all macromolecular species present. Generally, a substantially pure protein means that about 80% to 90% or more of the macromolecular species present in the composition is the purified species of interest. Methods to for determining protein purity in a composition are known in the art, and include for example, those described by Rhodes and Laue, Methods Enzymol. 2009, 463:677-89. The Dnd proteins (or homologs or equivalents thereof) may be recombinantly expressed from any of the isolated dnd genes (including dnd mutant genes), as described herein. In some aspects, the purified proteins may be further combined with substrates, co-factors and such salts, metal ions, buffers, etc., as may be required for enzyme activity. For example, the composition may further comprise e.g., L-cysteine as a sulfur donor, pyridoxal phosphate as a co-factor for Dnd enzyme activity, divalent metal ions, buffers to control pH, salts to control ionic strength, etc.

### Methods for Phosphorothioate (PS) Modification of Nucleic Acids

According to one embodiment, a method for incorporating PS modifications into the sugar-phosphate backbone of a nucleic acid is provided. The method involves transforming a bacterium which expresses a number of Dnd proteins (or homologs or equivalents thereof) sufficient to incorporate phosphorothioate modifications into a nucleic acid (e.g., as described herein), with a nucleic acid vector to be modified. In some aspects, in addition to the vector to be modified, the bacterium is transformed with one or more vectors encoding one or more Dnd gene products as described herein. The term “vector,” is a known term of art, and generally refers to a nucleic acid molecule capable of transforming a host, such as the bacteria provided herein. Vectors include, but are not limited to, plasmids, viral vectors, cosmids, artificial chromosomes, and phagemids. In one embodiment, the vector is one which is able to replicate in a host cell. Vectors may contain one or more marker sequences suitable for use in the identification and/or selection of cells which have or have not been transformed or genomically modified with the vector. Markers include, for example, genes encoding proteins which increase or decrease either resistance or sensitivity to antibiotics (e.g., kanamycin, ampicillin) or other compounds, genes which encode enzymes whose activities are detectable by standard assays known in the art (e.g., β-galactosidase, alkaline phosphatase or luciferase), and genes which visibly affect the phenotype of transformed or transfected cells, hosts, colonies, or plaques.

In some aspects, the vector or nucleic acid to be modified is large, as compared to those nucleic acids that are able to be modified by PS incorporation using current methodologies. For example, in some aspects, “large” encompasses nucleic acids which are genomes or as large as genomes, e.g., comprising millions to billions of base pairs. In some aspects, a large nucleic acid is a plasmid that is at least 500 bp in length, at least 1,000 base pairs (e.g., 1 kilobase (kb)), to at least 2 kb, at least 3 kb, at least 4 kb, at least 5 kb, at least 6 kb, at least 7 kb, at least 8 kb, at least 9 kb, at least 10 kb, at least 12 kb, at least 15 kb, at least 18 kb, at least 20 kb, at least 25 kb, or at least 30 kb or more in length. In some aspects, a large nucleic acid is an artificial chromosome (e.g., a bacterial artificial chromosome (BAC)) that is at least 50 kb in length, at least 75 kb, at least 100 kb, at least 150 kb, at least 200 kb, at least 250 kb, at least 300 kb, at least 400 kb, at least 500 kb, at least 600 kb, at least 700 kb, at least 800 kb, at least 900 kb, at least 1,000 kb, at least 1,500 kb, or at least 2,000 kb or more in length. In some aspects, a large nucleic acid is a linear nucleic acid, for example a linear DNA or RNA molecule of at least 100 bp, at least 200 bp, at least 300 bp, at least 400 bp, at least 500 bp, at least 1 kb, at least 2 kb, at least 3 kb, at least 4 kb, at least 5 kb, at least 8 kb, at least 10 kb, at least 12 kb, at least 15 kb, at least 18 kb, at least 20 kb, at least 25 kb, at least 30 kb, at least 50 kb, at least 75 kb, or at least 100 kb or more in length. It should be appreciated however, that small nucleic acid molecule may also be PS modified, for example nucleic acids as small as a dinucleotide (See Examples).

In some aspects, the bacteria transformed is any bacteria as described herein, e.g., bacteria expressing dnd genes (endogenously or as a result of being engineered to express the genes). In some aspects, the bacteria is E. coli. The E. coli may express endogenous dnd genes (e.g., Escherichia coli B7A), or may be genetically engineered to express any combination of dnd genes as described herein. Methods for genetic alterations of microbes are well known to those of skill in the art, and are described, for example, in J. Sambrook and D. Russell, Molecular Cloning: A Laboratory Manual, Cold Spring Harbor Laboratory Press; 3rd edition (Jan. 15, 2001). For example, the bacteria (e.g., E. coli) may be engineered to express the dnd genes from a plasmid encoding them, or the bacteria may have the dnd genes genomically incorporated, e.g., through recombineering methodologies as described in Sawitzke et al., Methods Enzymol. 2007, 421: 171-199.

In some aspects, the method further involves isolating the vector from the bacterium after an amount of time sufficient for the incorporation of PS modifications. Methods for isolating vectors from bacteria are well known in the art, and include for example, those described in Molecular Cloning: A Laboratory Manual, Cold Spring Harbor Laboratory Press; 3rd edition (Jan. 15, 2001). In some aspects, an “amount of time sufficient” for the incorporation of PS modifications is the amount of time necessary for at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% or more of the possible nucleotides to be modified. Methods for assessing the amount of PS modifications in a nucleic acid are known in the art, and include for example, those as described in Wang et al., Proc. Natl. Acad. Sci. 2011, 108(7):2963-8. For example, Escherichia coli B7A is known to incorporate PS modifications in GA (e.g., d(G_{PS}A)) and GT (e.g., d(G_{PS}T)) sequences, thus the amount of time sufficient to incorporate PS modifications is the amount of time necessary for at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% or more of GA and GT sequences to be modified.

In some aspects, the method involves adding substrates, co-factors and such salts, buffers, etc., as may be required for enzyme activity. For example, the method can further comprise contacting the bacteria with e.g., L-cysteine as a sulfur donor, and pyridoxal phosphate as a co-factor for Dnd enzyme activity.

According to another embodiment, a method for incorporating phosphorothioate modifications into the sugar-phosphate backbone of a nucleic acid is provided, which comprises contacting a nucleic acid with a composition comprising one or more Dnd proteins (or homologs or equivalents thereof), such as one or more proteins in a bacterial cell lysate or a combination of lysates, e.g., any lysate as provided herein. The nucleic acid is any nucleic acid as described herein, e.g., oligonucleotides, siRNAs, and the like. In some aspects, the composition being contacted with a nucleic acid, or the method comprises also contacting with a composition that, comprises substrates, co-factors and/or such salts, buffers, etc., as may be required for enzyme activity, as provided herein. For example, the composition can comprise e.g., L-cysteine as a sulfur donor, and pyridoxal phosphate as a co-factor for Dnd enzyme activity. In some aspects, the method further involves isolating the nucleic acid after an amount of time sufficient (e.g., as described herein) for the incorporation of phosphorothioate modifications. Methods for isolating a nucleic acid are well known in the art, and include, for example, centrifugation followed by chloroform/phenol extraction and ethanol precipitation.

According to another embodiment, a method for incorporating phosphorothioate modifications into the sugar-phosphate backbone of a nucleic acid is provided, which comprises contacting a nucleic acid with a composition comprising one or more purified Dnd proteins (or to homologs or equivalents thereof), e.g., any composition comprising Dnd proteins provided herein. The nucleic acid is any nucleic acid as described herein, e.g., oligonucleotides, siRNAs, and the like. In some aspects, the composition being contacted with a nucleic acid, or the method comprises contacting with a composition that, comprises substrates, co-factors and/or such salts, metal ions, buffers, etc., as may be required for enzyme activity, as provided herein. In some aspects, the method further involves isolating the nucleic acid after an amount of time sufficient (e.g., as described herein) for the incorporation of phosphorothioate modifications. Methods for isolating a nucleic acid are well known in the art, and include, for example, centrifugation followed by chloroform/phenol extraction and ethanol precipitation.

### Kits

According to other embodiments, kits for incorporating phosphorothioate modifications into the sugar-phosphate backbone of a nucleic acid are provided. A “kit,” as used herein, typically defines a package or an assembly including one or more of the compositions of the invention, and/or other compositions associated with the invention, for example, as described herein. In one embodiment, the kit comprises a transformable bacterium which expresses a number of Dnd proteins (or homologs or equivalents thereof) sufficient to incorporate phosphorothioate modifications into a nucleic acid, e.g., as described herein. The bacterium may be any bacterium provided herein, e.g., a bacterium expressing dnd genes. By “transformable,” it is meant that the bacterium is capable of being transformed with a vector (e.g., those described herein), using routine methods, such as electroporation or heat-shock transformation of cells first made chemically competent, for example as described in Molecular Biology: Current Innovations and Future Trends by Griffin A. M. and Griffin H. G. (1995) Horizon Scientific Press, Norfolk, U.K; and Modern Genetic Analysis by Griffith A. J., Second Edition, (2002) H. Freeman and Company, New York, N.Y.

In another embodiment, the kit comprises one or more compositions comprising one or more Dnd proteins (or homologs or equivalents thereof) sufficient to incorporate phosphorothioate modifications into a nucleic acid (e.g., as described herein) alone or in combination with other Dnd proteins (or homologs or equivalents thereof), such as one or more bacterial lysates, one or more Dnd proteins, etc. In some aspects, the kit can include one or more suspensions comprising any Dnd protein (e.g., purified Dnd protein) (or homolog or equivalent thereof) provided herein. A “suspension,” as used herein, represents an aqueous to solution which comprises one or more soluble proteins, e.g., one or more Dnd proteins provided herein. In some aspects, the kit comprises a single suspension comprising one or more Dnd proteins (or homologs or equivalents thereof). In some aspects, the kit comprises a number of suspensions, each suspension comprising one or more Dnd proteins (or homologs or equivalents thereof). In some aspects, the kit includes one or more compositions of one or more Dnd proteins (or homologs or equivalents thereof) that are lyophilized In some aspects, the kit comprises a single lyophilized composition comprising one or more Dnd proteins (or homologs or equivalents thereof). In some aspects, the kit comprises a number of lyophilized compositions, each comprising one or more Dnd proteins (or homologs or equivalents thereof). In some aspects, the kit further comprises substrates, co-factors and such salts, metal ions, buffers, etc., as may be required for enzyme activity. For example, the kit can further comprise e.g., L-cysteine as a sulfur donor, pyridoxal phosphate as a co-factor for Dnd enzyme activity, divalent metal ions, buffers to control pH, salts to control ionic strength, etc.

In yet another embodiment, the kit comprises one or more vectors comprising one or more dnd genes, for example any one or combination of dnd genes as provided herein. As used herein, “dnd gene” is intended to include the full-length gene or any portion thereof that is capable of expressing the protein. It is to be understood that a vector comprising one or more dnd genes, is a vector that is able to transform a host cell, such as E. coli, that the vector is able to replicate in the host cell, and is able to express functional Dnd proteins (or homologs or equivalents thereof) therefrom, e.g., as described herein. Any vector suitable for the transformation of E. coli may encode or comprise a dnd gene, e.g., as described herein, for example vectors belonging to the pUC series, pGEM series, pET series, pBAD series, pTET series, or pGEX series. In some aspects, the vector is a bacterial artificial chromosome (BAC). In some aspects, the kit further comprises substrates, co-factors and such salts, buffers, etc., as may be required for enzyme activity and/or transformation of cells. For example, as described herein. In some aspects, the kit further comprises competent cells.

The invention also provides kits that include containers of the compounds or compositions described herein. It is contemplated that the compounds or compositions may be supplied as a “kit-of-parts” comprising the compound, composition, or subpart thereof in one container and an amount of a compound, composition, or subpart thereof, or a carrier in a second container and, optionally, one or more suitable diluents for the foregoing components in one or more separate containers. In these embodiments, the compounds, subparts, carriers, or other molecules may be supplied in a concentrated form (e.g., a concentrated Dnd protein and/or enzyme co-factors and substrates), such as a concentrated aqueous solution. It may even be supplied in frozen form or in freeze-dried or lyophilized form. The latter may be more stable for long term storage and may be defrosted and/or reconstituted with a suitable diluent immediately prior to use. For example, a kit can include one or more containers that contain bacteria as provided herein. In other aspects, a kit can include one or more containers that contain one or more Dnd proteinss(or homologs or equivalents thereof), as described herein (e.g., in an aqueous solution or lyophilized form). In yet other aspects, the kit can include one or more containers that contain one or more vectors encoding one or more dnd genes, as provided herein. In some aspects, a kit can include one or more containers that contain substrates, co-factors and such salts, buffers, etc., as may be required for enzyme activity. For example, as described herein.

In some embodiments, the container(s) can each be a vial, bottle, ampoule or bag. In some embodiments, therefore, the composition(s) of the kit can each be in a vial, bottle, ampoule or bag. In some embodiments, all the compositions of the kit can each be together in a vial, bottle, ampoule or bag, or some combination thereof.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the composition and/or other compositions associated with the kit. The instructions may be provided in any form recognizable by one of ordinary skill in the art as a suitable vehicle for containing such instructions, for example, written or published, verbal, audible, digital, optical, visual (e.g., videotape, DVD, etc.) or electronic communications (including Internet or web-based communications), provided in any manner.

### EXAMPLES

The present invention will be more specifically illustrated by the following examples. However, it should be understood that the present invention is not limited by these examples in any manner.

### Experimental Design

The following describes exemplary methods for in vitro phosphorothioation of DNA using Salmonella enterica serovar Cerro 87, which possesses a well-characterized PT modification system comprised of IscS protein in place of DndA and Dnd proteins B, C, D and E.¹ DndB is a transcriptional regulator that is not required for this PT reaction.¹It has been demonstrated that the S. enterica proteins catalyze incorporation of PT into the d(G_{PS}A) and d(G_{PS}T) contexts at a 1:1 ratio, which suggested that the consensus sequence for modification involved GAAC on one strand and GTTC on the other, as a palindromic PT consensus sequence.¹ This consensus sequence was built into the oligodeoxynucleotide constructs used as substrates for the in vitro PT modification reactions. In all cases, the oligodeoxynucleotides were annealed with their corresponding complementary stands, followed by immobilization on the streptavidin-coated Dynabeads. This complex was incubated with either cell-free extracts or a mixture of purified proteins, with the resulting PT modifications analyzed using an established LC-MS/MS method that quantifies PT-containing nucleic acids.¹

### Materials and Methods

Bacterial Strains, Culturing Conditions and Plasmids

Salmonella enterica serovar Cerro 87 and its derivative mutants were used for cell-free extract isolation. pET28a, pET-15b and E. coli BL21(DE3,plusE) (Novagen) were used for heterologous over-expression of proteins. All the strains were cultured in Luria-Bertani (LB) broth at 37° C.

### Preparation of Cell Extracts (Bacterial Cell Lysates)

One form of the in vitro phosphorothioation assay was performed using cell-free extracts isolated from Salmonella enterica serovar Cerro 87, which expresses the four proteins necessary for PT incorporation into DNA: iscS (equivalent to dndA) and dnd C-E. To prepare extracts, bacteria were grown in 200 mL of LB medium at 37° C. to an A₆₀₀ of 1.5 and then harvested by centrifugation at 5000×g for 10 mM at 4° C. Cells were washed three times with PBS (137 mM NaCl, 2.7 mM KCL 10 mM Na₂HPO₄, 2 mM KH₂PO₄, pH7.4), and finally resuspended in 20 mL of lysis buffer (20 mM Tris-HCl, pH 8.0, 60 mM KCl, 10 mM Mg₂Cl, 1 mM EDTA, 2 mM DTT, 1 mM PMSF and 25% glycol). Resuspended bacteria were subjected to three complete cycles of freezing at −80° C. and rapid thawing to 0° C., and then to probe sonication until no intact cells were visible by light microscopy. The soluble protein from the cell lysate was collected as the supernatant following centrifugation at 15000×g for 20 mM at 4° C. The crude extract was used immediately for phosphorothioation reactions with DNA substrates prepared as described shortly.

### Preparation of Recombinant Dnd Proteins

Another form of the in vitro phosphorothioation assay involves the use of purified forms of the S. enterica serovar Cerro 87 Dnd proteins needed to incorporate PT into DNA: IscS and DndC-E. The expression and purification of these proteins involves cloning of individual genes into an expression vector and purification of individual proteins from cells transformed with the expression vector. The iscS gene from S. enterica serovar Cerro 87 genomic DNA, with flanking NdeI and BamHI restriction sites, was amplified with primers iscS-A (5′GGTGAATTCTTAATGATGTGCCCATTCGAT3′; EcoRI sites underlined)(SEQ ID NO: 1) and IscS-S (5′GGGCATATGAAATTACCGATTTATCTC3′; NdeI sites underlined)(SEQ ID NO: 2). After restriction endonuclease digestion, the insert was ligated into same endonuclease digested pET-15b with a hexahistidine-tag at the N terminus of iscS to create a His-tagged IscS expression vector. In a similar manner, the three contiguous genes dndC, dndD and dndE were amplified as a single block from S. enterica serovar Cerro 87 genomic DNA using the following primers: Dnd-A (5′TTGCCATATGAGTAAATTAGTTCAGGC 3′; NdeI sites underlined)(SEQ ID NO: 3) and Dnd-S (5′CGCGGATCCTATGGCACCGTTCATGGTGC3′; BamHI sites underlined)(SEQ ID NO: 4). After restriction endonuclease digestion, this block of three genes was cloned into pET-28a with a hexahistidine-tag at the N terminus of DndC; DndD and DndE are known to bind to DndC, so three are co-purified using the DndC His-tag.

Both of the expression vectors were used to transform BL21(DE3) cells (Novagen) to express the His-tagged proteins. An overnight culture of cells was used to inoculate 1 L of LB medium containing corresponding antibiotics and the culture was grown to an A₆₀₀ of 0.6. Expression was induced by adding IPTG to a final concentration of 0.2 mM and growth continued for 5 h at 30° C. Cells were harvested and stored at −70° C. The frozen cell pellets from 1 L cultures were resuspended in 50 mL of 50 mM Tris-HCl, pH 7.0, 500 mM NaCl, and 1 mM DTT, and the cells lysed by probe sonication. The soluble fraction (supernatant) was collected by centrifugation (15000×g, 30 min at 4° C.) and loaded onto a HisTrap HP column (GE Healthcare, 1 mL). The proteins were eluted with a linear gradient of buffer B (50 mM Tris-HCl, pH 8.0, 150 mM NaCl, 500 mM imidazole) with an AKTA Fast Protein Liquid

Chromatography system (GE Healthcare). Eluted fractions were analyzed by SDS-PAGE and the His-tagged proteins were concentrated by centrifugal filtration (Millipore, 30 kDa). Subsequent desalting was achieved by buffer exchange with desalting buffer (50 mM Tris-HCl, pH 7.0, 150 mM NaCl). The purified proteins were then used in the in vitro phosphorothioation reactions with DNA substrates prepared as described.

### Preparation of Duplex DNA Substrates for the Phosphorothioation Reactions

The oligonucleotide substrates used in this study are listed in Table 1. To prepare duplex oligonucleotides, 100 μL of 100 pmol/μL aqueous solutions of each complementary biotinylated oligonucleotides were mixed a 1.5 ml microfuge tube and heated at 95° C. for 5 min, followed by cooling to ambient temperature. The annealed biotinylated oligonucleotides were then linked to streptavidin-coated beads. A volume of 100 μl of Dynabead M-280 Streptavidin (Invitrogen) was washed for three times in 1 mL PBS, and 10 μL of 10 pmol/μL duplex biotinylated oligonucleotide was added, with incubation for 30 min at 25° C. The oligonucleotide-bead conjugates were washed three times with 1 mL PBS.

### In Vitro Phosphorothioation of DNA Using Cell-Free Extracts

The standard reaction conditions for in vitro phosphorothioation using cell extracts were as follows: final volume, 200 μL; 100 pmol (100 μL) of duplex biotinylated oligonucleotides bound to Dynabead M-280 Streptavidin; 2.5 mM ATP; 1 mM L-cysteine, 0.1 mM pyridoxal phosphate and 1 mL cell extract. The reaction mixture was incubated at 25° C. for 2 h and terminated by washing the beads three times in 1 mL PBS using a Dynal magnet to immobilize the beads. Samples were immediately processed by enzymatic hydrolysis and LC-MS/MS analysis of PT-containing dinucleotides, as described below.

### In Vitro Phosphorothioation of DNA Using Purified Recombinant Proteins

The in vitro DNA phosphorothioation assays with purified proteins were carried out using the following conditions: final volume, 500 μL; 1 μg of DNA substrate; 10 mM Tris-HCl, pH 7.5, 50 mM KCl, 2.5 mM ATP, 1mM L-cysteine, 0.1 mM pyridoxal phosphate, 10 mM MgCl₂, 2 μg of IscS protein and 10 μg of purified DndC/D/E complex. The reactions were performed at 25° C. for 1 h and then terminated by addition of 1:1 volume 1:1 to phenol:chloroform. Following extraction, the aqueous fraction was removed and DNA products were precipitated with 2 volumes of ethanol, followed by centrifugation at 16,000×g and washing of the pellet with 70% ethanol. The pellets were redissolved in buffer for subsequent hydrolysis and LC-MS/MS analysis of PT-containing dinucleotides.

### Enzymatic Hydrolysis of Bead-Bound Oligonucleotides

Samples of eluted oligodeoxynucleotide were hydrolyzed in a 200-μL volume with 4 U nuclease P1, 30 mM sodium acetate, pH 5.2, 0.5 mM ZnCl₂ at 50° C. for 2 h. Subsequent dephosphorylation was carried out by addition of 20 μL of 1 M Tris-Cl, pH 8.0, and 17 U of alkaline phosphatase at 37° C. for another 2 h. The enzymes were subsequently removed by ultrafiltration (YM-10 column; Microcon). The PT-containing dinucleotides d(G_{PS}A) and d(G_{PS}T) were quantified by LC-MS/MS.

### LC-MS/MS Analysis of Phosphorothioation-Containing Dinucleotides

The PT-containing dinucleotides d(G_{PS}A) and d(G_{PS}T) was quantified using an HPLC-coupled Agilent 6410 Triple Quad (QQQ) mass spectrometer. Chromatographic resolution was achieved using a Agilient ZORBAX SB-C18 column (150×2.1 mm, 3.5 μm bead size) with elution (35° C., 0.3 mL/min) using a gradient of 97% buffer A (0.1% acetic acid in water) and 3% buffer B (0.1% acetic acid in acetonitrile) for 5 min, followed by 3-15% buffer B over 20 min, and 15-100% buffer B over 1 min. The eluent was analyzed by QQQ using an electrospray ionization source in positive mode with the following parameters: gas flow, 10 L/min; nebulizer pressure, 30 psi; drying gas temperature, 325° C.; and capillary voltage, 3100 V. Multiple reaction monitoring mode was used for detection of product ions derived from the precursor ions, with all instrument parameters optimized for maximal sensitivity (retention time in min, precursor ion m/z, product ion m/z, fragmentor voltage, collision energy): d(G_{PS}A), 20.5, 597, 136, 120 V, 40 V; d(G_{PS}T), 26.5, 588, 152, 110 V, 17 V. Quantification was achieved using an external calibration curves prepared with synthetic standards.

TABLE 1
Oligodeoxynucleotides used in this study
(complementary strands not shown)

| | |
|---|---|
| dpt101 | 5′-Biotin-GTCGTGGTTGGCGACG_{PS}AACACC |
| | AGACCGTTA-3′ (SEQ ID NO: 5) |
| dpt102 | 5′-Biotin-GTCGTGGTTGGCGACGAACACCAG |
| | ACCGTTA-3′ (SEQ ID NO: 6) |
| dpt104 | 5′-Biotin-GTCGTGGTTGGCGACGTACACCAG |
| | ACCGTTA-3′ (SEQ ID NO: 7) |
| dpt105 | 5′-Biotin-GTCGTGGTTGGCGAAGAACGCCAG |
| | ACCGTTA-3′ (SEQ ID NO: 8) |
| dpt106 | 5′-Biotin-GCGGGGAAAGCCGGCGAACGTGGC |
| | GAGAAAG-3′ (SEQ ID NO: 9) |
| dpt107 | 5′-Biotin-GGCGAAC-3′ |
| dpt113 | 5′-Biotin-GCGGGGAAAGCCGGCG_{PS}AACGTGG |
| | CGAGAAAG-3′ (SEQ ID NO: 10) |
| dpt114 | 5′-Biotin-GCTTTCTCGCCACG_{PS}TTCGCCGGC |
| | TTTCCCCG-3′ (SEQ ID NO: 11) |
| dpt116 | 5′-Biotin-CTCGCTCACTGACTCGCTGCGCTC |
| | GGTCGTTCGGCTGCGGCGAGCGGTATCAGCTCAC |
| | TC-3′ (SEQ ID NO: 12) |
| dpt117 | 5′-Biotin-TCAAGGATCTTACCGCTGTTGAGA |
| | TCCAGTTCGATGTAACCCACTCGTGCACCCAACT |
| | GA-3′ (SEQ ID NO: 13) |

### Example 1

### In Vitro Phosphorothioation of Double-Stranded Oligonucleotides with S. enterica Serovar Cerro 87 Sell-Free Extracts

The first system developed involved use of S. enterica extracts, which contain IscS and Dnd proteins C-E, with a duplex oligodeoxynucleotide substrate (a 5′-terminal biotinylated 30 by oligodeoxynucleotide, dpt102; Table 1) bound to magnetic beads to facilitate processing. As a positive control for the presence of PT in the expected d(GpsA) and d(GpsT) sequence contexts, an oligodeoxynucleotide substrate (dpt101, Table 1) containing PT modifications was synthesized. To ensure that genomic DNA from the bacteria did not contaminate the extract and contribute to the detected level of PT modifications, control experiments were performed with reactions lacking oligodeoxynucleotide substrates and with reactions using cell extracts prepared from an S. enterica mutant lacking the dnd genes (XTG103).²

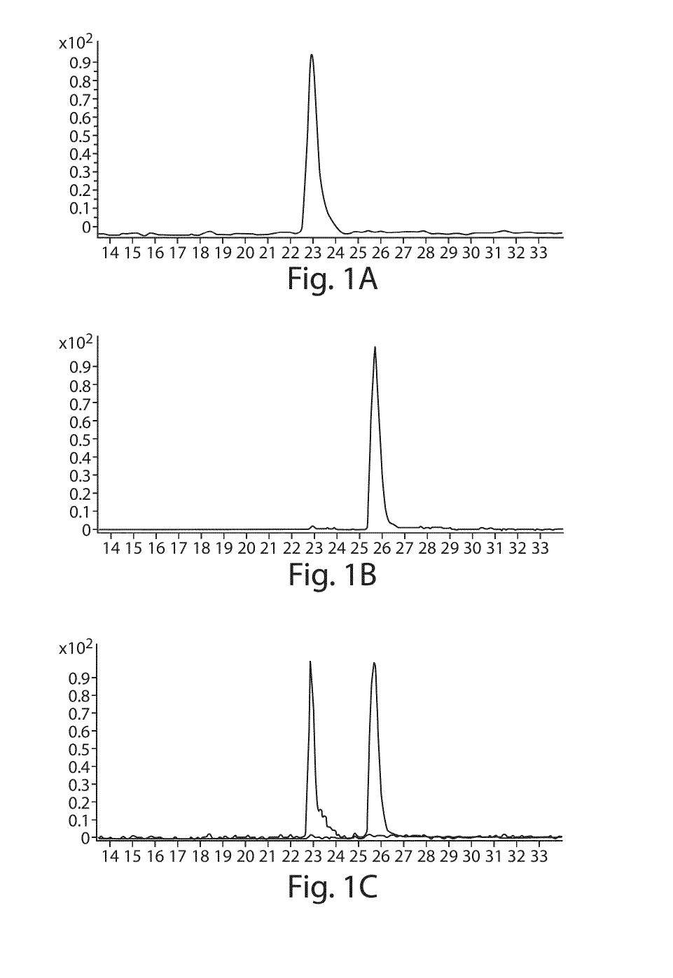

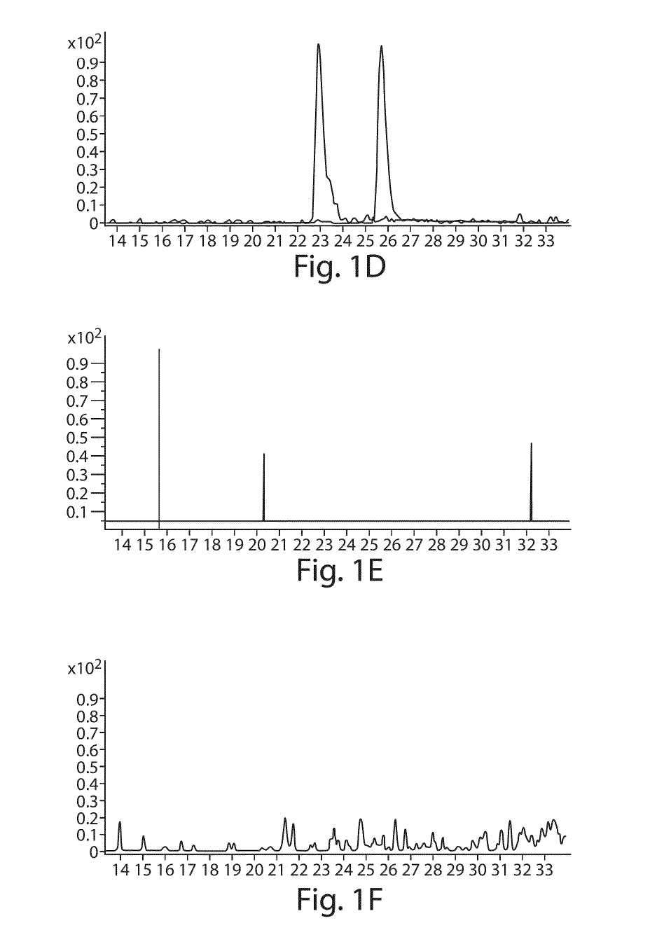

The results of the reactions with cell-free assays are shown in FIG. 1. The absence of PT-containing dinucleotides in the negative control samples confirmed that all cellular PT-containing DNA had been removed from the reaction mixtures (FIG. 1E,F). Under the same conditions, the cell-free extract prepared from wild-type S. enterica reacted with the dpt102 substrate to form PT modifications in d(G_{PS}A) and d(G_{PS}T) sequence contexts, consistent with the positive control oligo dpt101, which indicated that PT modifications were incorporated in the in vitro reaction with the cell-free extract, which is consistent with the S. enterica genomic DNA analysis performed previously.¹

### Example 2

### Defining the PT Consensus Sequence Using In Vitro Phosphorothioation with Cell-Free Extracts

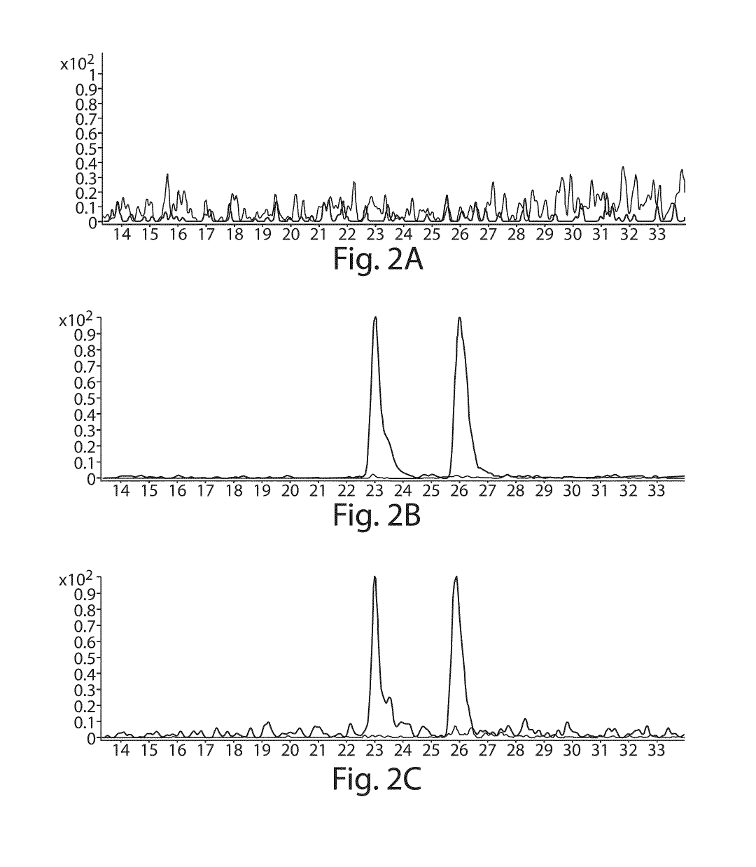

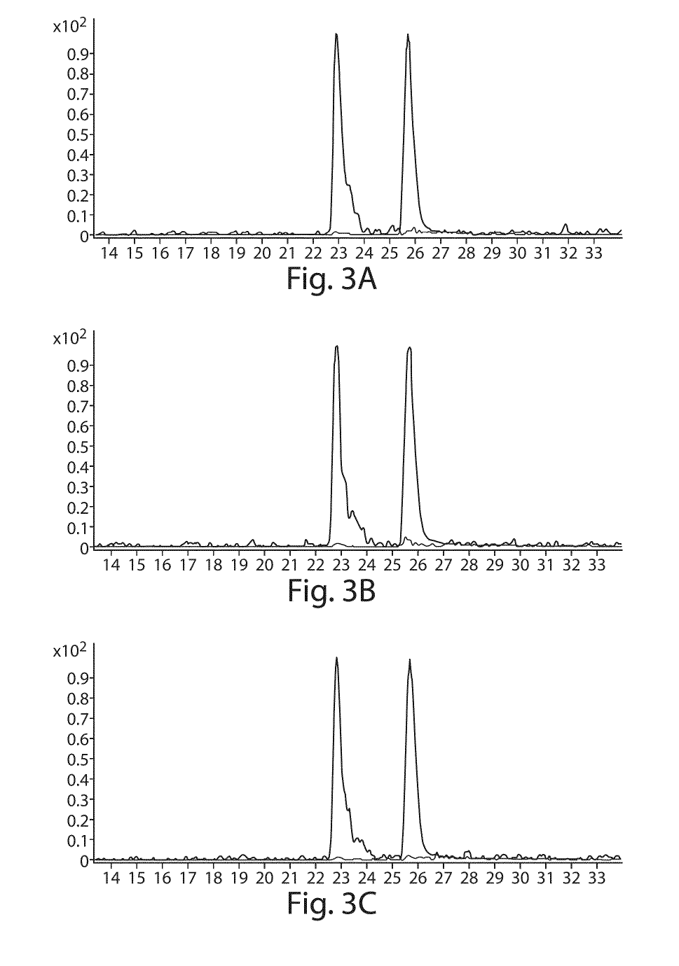

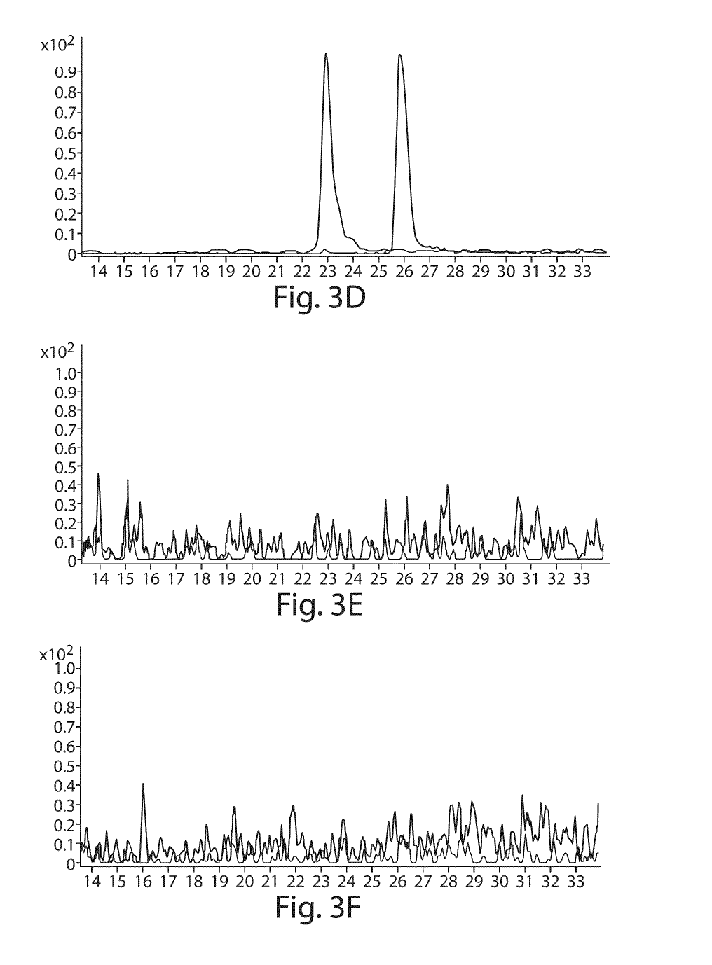

The cell-free phosphorothioation system was first applied to assess the putative GAAC/GTTC recognition sequence of the S. enterica Dnd proteins. The role of internal sequence was tested using the dpt104 substrate in which the putative modification motif (GAAC/GTTC) was changed to GTAC/GTAC (Table 1). As shown in FIG. 2A, this resulted in loss of detectable PT dinucleotides and confirmed G_{PS}AAT/G_{PS}TTC as the central recognition element for S. enterica. Given the potential for 5- to 6-nt consensus sequence based on PT frequency in the S. enterica genome,¹ the two flanking nucleotides in substrate dpt102 were altered (5′-cGAACa-3′) to 5′-aGAACg-3′ (dpt105; Table 1), with matching changes in the complementary strand. LC-MS analysis showed that dpt105 was also an efficient substrate for in vitro phosphorothioation by extracts (FIG. 2C), which confirms a four-nucleotide GAAC/GTTC consensus for S. enterica Dnd proteins. The role of flanking sequence was further tested using a 30 bp substrate (dpt106) in which the sequence flanking GAAC/GTTC was significantly altered (Table 1). Again, these changes had no effect on phosphorothioation of the substrate, as shown in FIG. 3A. The role of substrate length was assessed using substrate dpt106 in which the 3′-flanking sequence was deleted (Table 1). As shown in FIG. 3B, the shorter substrate was still phosphorothioated at the expected site in GAAC/GTTC.

### Example 3

### In Vitro Phosphorothioation of Partially Modified Sites in DNA

As a post-replication modification, DNA phosphorothioation has been proposed to serve as a restriction-modification system, thus suggesting that the pattern of phosphorothioation is clonally inherited in a semi-conservative manner, with Dnd proteins catalyzing PT modification in a consensus sequence already possessing a PT modification on one strand. To test this hypothesis, two hemi-phosphorothioated substrates were designed (dpt 113, dpt114; Table 1) containing a single PT modification on one strand or the other. As expected, the cell-free extract was capable of inserting PT into the unmodified strands of the two hemi-modified substrates (FIG. 3C,D). However, single-strand substrates (i.e., the unmodified complementary strands of dpt 113 and dpt 114) were not substrates for the Dnd proteins (FIG. 3E,F).

### Example 4

### In Vitro Reconstitution of DNA Phosphorothioation by Recombinant Dnd Proteins

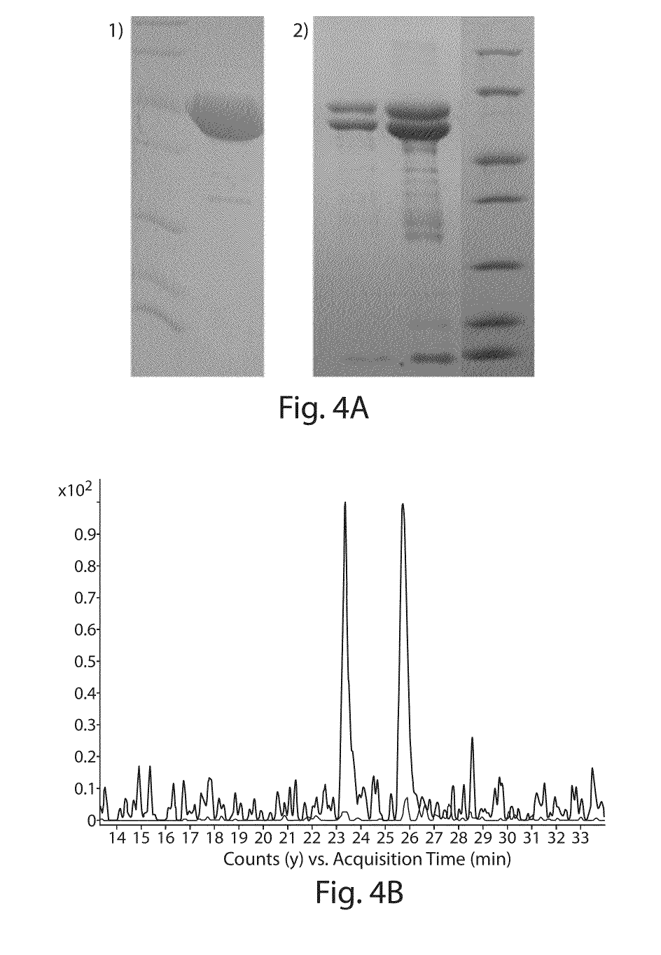

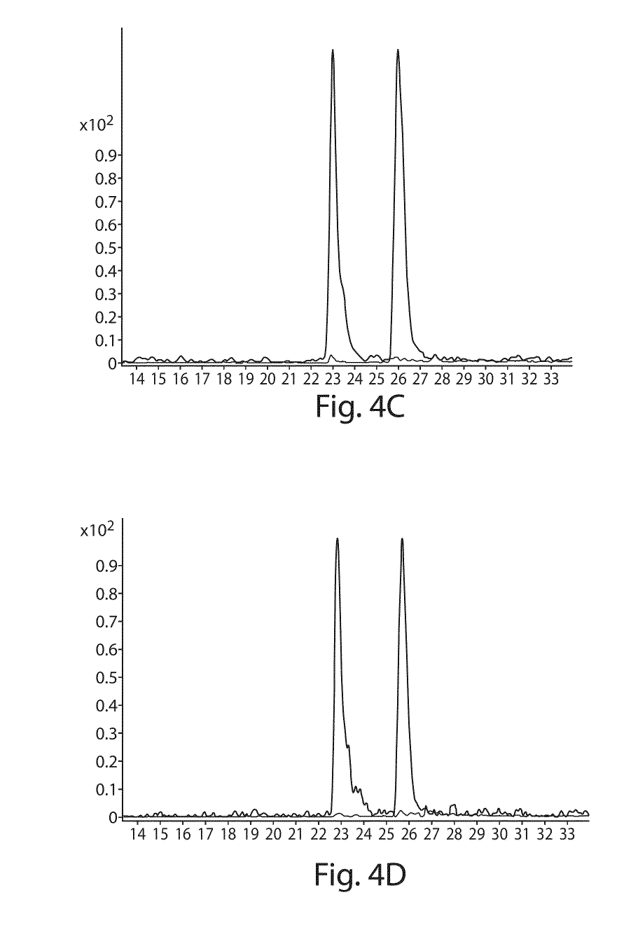

The in vitro phosphorothioation system was further refined by replacing the cell-free extract with purified proteins to reconstitute functional PT synthesis. This was accomplished using a mixture of the purified His-tag-labeled DndCDE complex and the His-tag-labeled IscS protein in reactions with L-cysteine as the sulfur donor,³ pyridoxal phosphate as a cofactor and to either phosphorothioate-free pBluescript SK+ plasmid DNA or two 60 bp double-stranded oligodeoxynucleotides containing GAAC/GTTC (dpt116, dpt117) as substrates (Table 1). As shown in FIG. 4, PT modifications in the expected dinucleotide sequence contexts were efficiently catalyzed in all of these substrates.

### References

1. Wang, L. et al. DNA phosphorothioation is widespread and quantized in bacterial genomes. Proc Natl Acad Sci 108, 2963-2968 (2011).
2. Xu, T., Yao, F., Zhou, X., Deng, Z. & You, D. A novel host-specific restriction system associated with DNA backbone S-modification in Salmonella. Nucleic Acids Res 38, 7133-41 (2010).
3. An, X. et al. A novel target of IscS in Escherichia coli: participating in DNA phosphorothioation. PLoS One 7, e51265 (2012).

### Equivalents and Scope

Those skilled in the art will recognize or be able to ascertain using no more than routine experimentation many equivalents to the specific embodiments described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be to modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention can be excluded from any one or more claims, for any reason, whether or not related to the existence of prior art.

All references cited herein, including patents, published patent applications, and publications, are incorporated by reference in their entirety.

What is claimed is:

1. A method for incorporating phosphorothioate modifications into the sugar-phosphate backbone of a nucleic acid, comprising
transforming a bacterium which expresses one or more Dnd proteins that incorporate phosphorothioate modifications into a nucleic acid, with a nucleic acid vector to be modified, and
isolating the vector from the bacterium.

2. The method of claim 1, wherein the bacterium is Escherichia coli.

3. The method of claim 1, wherein the bacterium expresses the Dnd protein(s) from either one or more vectors, a plasmid or from the bacterium's genomic DNA.

4. The method of claim 1, wherein the method also comprises transforming the bacterium with one or more vectors or a plasmid that encode(s) one or more Dnd protein(s).

5. The method of claim 1, wherein the bacterium expresses DndA (or IscS), DndC, DndD, and DndE.

6. The method of claim 1, wherein the bacterium expresses DndA (or IscS), DndB, DndC, DndD, and DndE.

7. The method of claim 1, wherein the Dnd protein(s) are expressed from dnd genes isolated from a genus selected from the group consisting of Bacillus, Burkholderia, Candidatus Methanoregula, Candidatus Pelagibacter, Citrobacter, Clostridium, Desulfatibacillum, Enterobacter, Escherichia, Exiguobacterium, Geobacter, Hahella, Mesorhizobium, Mycobacterium, Oceanobacter, Pseudoalteromonas, Pseudomonas, Roseobacter, Salmonella, Shewanella, Streptomyces, Vibrio, and combinations thereof.

8. The method of claim 1, wherein the Dnd protein(s) are expressed from dnd genes isolated from a species selected from the group consisting of Streptomyces lividans, Salmonella enterica, Pseudomonas fluorescens, Escherichia coli, and combinations thereof.

9. The method of claim 1, wherein the bacterium is contacted with Dnd enzyme co-factors and/or substrates.

10. The method of claim 1, wherein the vector is a plasmid between 1 and 25 kb in length.

11. The method of claim 1, wherein the phosphorothioate modifications are of the Rp stereo-isoform configuration.

12. The method of claim 1, wherein the vector is isolated after an amount of time sufficient for the incorporation of phosphorothioate modifications.

13. A composition for incorporating phosphorothioate modifications into the sugar-phosphate backbone of a nucleic acid, comprising one or more bacterial lysates, each lysate comprising one or more Dnd proteins that incorporate phosphorothioate modifications into a nucleic acid.

14. The composition of claim 13, wherein the bacterial lysate(s), alone or in combination, comprise DndA (or IscS), DndC, DndD, and DndE.

15-18. (canceled)

19. A composition for incorporating phosphorothioate modifications into the sugar-phosphate backbone of a nucleic acid, comprising one or more purified Dnd proteins that incorporate phosphorothioate modifications into a nucleic acid.

20-24. (canceled)

25. A method for incorporating phosphorothioate modifications into the sugar-phosphate backbone of a nucleic acid, comprising
contacting a nucleic acid with a composition of claim 13, and isolating the nucleic acid.

26. (canceled)

27. A kit for incorporating phosphorothioate modifications into the sugar-phosphate backbone of a nucleic acid, comprising the composition of claim 13, and one or more containers and/or directions for use.

28. A kit for incorporating phosphorothioate modifications into the sugar-phosphate backbone of a nucleic acid, comprising
a transformable bacterium which expresses one or more Dnd proteins that incorporate phosphorothioate modifications into a nucleic acid, and one or more containers and/or directions for use.

29-37. (canceled)

38. A kit for incorporating phosphorothioate modifications into the sugar-phosphate backbone of a nucleic acid, comprising
one or more purified Dnd protein(s) that incorporate phosphorothioate modifications into a nucleic acid, and one or more containers and/or directions for use.

39-44. (canceled)

45. A kit for incorporating phosphorothioate modifications into the sugar-phosphate backbone of a nucleic acid, comprising one or more vectors each comprising one or more dnd genes, and one or more containers and/or directions for use.

46-51. (canceled)


SEQUENCE LISTING (13 sequences)

SEQ ID NO: 1 - DNA, 30 bp, artificial sequence
  primer
ggtgaattcttaatgatgtgcccattcgat

SEQ ID NO: 2 - DNA, 27 bp, artificial sequence
  primer
gggcatatgaaattaccgatttatctc

SEQ ID NO: 3 - DNA, 27 bp, artificial sequence
  primer
ttgccatatgagtaaattagttcaggc

SEQ ID NO: 4 - DNA, 29 bp, artificial sequence
  primer
cgcggatcctatggcaccgttcatggtgc

SEQ ID NO: 5 - DNA, 31 bp, artificial sequence
  oligo
gtcgtggttggcgacgaacaccagaccgtta

SEQ ID NO: 6 - DNA, 31 bp, artificial sequence; identical to SEQ ID NO: 5
  oligo

SEQ ID NO: 7 - DNA, 31 bp, artificial sequence
  oligo
gtcgtggttggcgacgtacaccagaccgtta

SEQ ID NO: 8 - DNA, 31 bp, artificial sequence
  oligo
gtcgtggttggcgaagaacgccagaccgtta

SEQ ID NO: 9 - DNA, 31 bp, artificial sequence
  oligo
gcggggaaagccggcgaacgtggcgagaaag

SEQ ID NO: 10 - DNA, 31 bp, artificial sequence; identical to SEQ ID NO: 9
  oligo

SEQ ID NO: 11 - DNA, 31 bp, artificial sequence
  oligo
gctttctcgccacgttcgccggctttccccg

SEQ ID NO: 12 - DNA, 60 bp, artificial sequence
  oligo
ctcgctcactgactcgctgcgctcggtcgttcggctgcggcgagcggtatcagctcactc

SEQ ID NO: 13 - DNA, 60 bp, artificial sequence
  oligo
tcaaggatcttaccgctgttgagatccagttcgatgtaacccactcgtgcacccaactga